(19) United States
(12) Patent Application Publication
(10) Pub. No.: US 2008/0118458 A1
(43) Pub. Date: May 22, 2008

(54) Agent containing L-Carnitine or L-Carnitine derivatives and at least one other specific substance

(76) Inventors: Melanie GIESEN, Geldern (DE); Klaus Rudolf SCHROEDER, Mettmann (DE); Dirk PETERSOHN, Koln (DE)

Correspondence Address: PAUL & PAUL, 2000 MARKET STREET, PHILADELPHIA, PA, 19103-3229, US

(21) Appl. No.: 11/970,187

(22) Filed: Jan. 7, 2008

Related U.S. Application Data
(63) Continuation of application No. PCT/EP2006/006193, filed on Jun. 27, 2006.

(30) Foreign Application Priority Data
Jul. 5, 2005 (DE) 10 2005 031 705.7

(51) Int. Cl. A61K 8/97; A61K 31/185; A61K 31/22; A61K 36/28

(52) U.S. Cl. 424/74; 424/737; 514/553; 514/546

(57) ABSTRACT

Compositions, in particular, for the treatment of the hair or of the skin, comprising at least one compound which is selected from L-carnitine or L-carnitine derivatives and at least one further substance which is selected from the group formed from active ingredients obtainable from the plants of the genus Echinacea, and also taurine and derivatives thereof.

### CROSS-REFERENCE TO RELATED APPLICATIONS

This application is a continuation under 35 U.S.C. Section 365(c) and 35 U.S.C. Section 120 of International Application No. PCT/EP2005/004079, filed Jun. 27, 2006. This application also claims priority under 35 U.S.C. Section 119 of German Patent Application No. DE 10 2005 031 705.7, filed Jul. 5, 2005. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention relates to compositions, in particular, for the treatment of the hair or of the skin, comprising at least one compound which is selected from L-carnitine or L-carnitine derivatives and at least one further substance which is selected from the group formed from active ingredients obtainable from the plants of the genus Echinacea, and also taurine and derivatives thereof.

(2) Description of Related Art, Including Information Disclosed Under 37 C.F.R. Sections 1.97 and 1.98.

L-carnitine is a vitamin-like active ingredient which is related to the B complex vitamins and is of essential importance for energy production and the metabolism of fat in the human body. For this reason, L-carnitine is used primarily as a food supplement (US 20040170709 A1). Like L-carnitine, L-carnitine tartrate is also used as a food supplement, e.g., for aiding weight reduction (US 20040028668 A1).

The human skin with its appendages is an organ of very complex structure which consists of a large number of different cell types. Each living cell of this organ is able to react to signals of its internal and external environment. These reactions of the cells are realized by controlled regulation on a gene and protein level, meaning that the metabolism of cells in the skin and its appendages is not static but very dynamic. However, the reactions of the skin and/or its appendages to changes in the surroundings must not be regarded as reactions of individual, isolated cells. Rather, each cell is integrated into a complex communication network. This network includes, for example, the communication between cells in the epidermis and cells in the dermis. Signal molecules such as, for example, interleukins, growth factors (e.g., KGF, EGF or FGF) etc. are involved in the communication between the cells in the skin and/or its appendages.

The aging process is a basic biological process which can be found in virtually all living organisms. Accordingly, the human skin is also affected by this phenomenon. Skin aging is a progressive process which leads to a loss in skin homeostasis. It is influenced by endogenous and exogenous factors. Whereas the endogenous aspects proceed as a “genetically controlled program,” environmental influences, such as, for example, UV light, are responsible for the exogenous factors.

ATP (adenosine triphosphate) is the universal storage form of chemical energy in cells. When the distal phosphate group cleaves off, ADP and Pi (inorganic phosphate) form. This reaction is highly exergonic, i.e., energy is released. ATP is produced upon cellular, oxidative degradation of fats, carbohydrates and proteins. ATP serves the cell, also the biologically active cells of the hair follicle, as energy supplier for biochemical syntheses and transport processes. These processes are endergonic, i.e., they proceed only with the input of energy. In order to optimally maintain and renew their metabolism and cellular structures, cells are thus reliant on an adequate supply of ATP. Thus, for example, the proliferation and differentiation of ORS keratinocytes (“keratinocytes of the outer root sheath”) is linked to the ATP synthesis, since for both processes the biosynthesis of specific proteins is an essential prerequisite. Dermal papillae cells also require ATP, for example, for the production of growth factors and thus for controlling the hair cycle. An adequate supply of the hair follicle with ATP is, therefore, an essential prerequisite for strong, vital and healthy hair.

The amount of a hair-active ingredient which can usually penetrate transdermally and specifically, transfollicularly as far as the hair bulb, is extremely small and depends essentially on the physiochemical properties of the substance itself (e.g., size, charge, lipophilicity) and also on the choice of formulation.

Hair follicle cells are subject to a genetically determined cycle of growth, regression and resting phase. The hair follicle is, therefore, the only organ which automatically renews itself continuously and thus has a unique metabolism depending on the particular growth phase. Thus, the metabolism of the hair follicle in the resting phase virtually comes to a standstill and is likewise reinitiated with each new start of a further cycle.

This cycle is controlled by a small, highly specialized cell population in the hair bulb, the dermal papillae cells, which control hair growth through a complex set of molecular signals which is specific for each phase of the hair cycle (Botchkarev V A et al. (2003) J Investig Dermatol Symp Proc 8:46-55).

The genus of the sun hat plants (Echinaceae) includes North American shrubs, some of which have already been used for a long time for aiding the treatment of infections (internal) and wounds (external). As are known, Echinacea extracts are used for stimulating the immune system and as antimicrobial and antiviral substance.

### BRIEF SUMMARY OF THE INVENTION

Surprisingly, it has been found that, through the topical use of compositions comprising L-carnitine or L-carnitine derivatives and at least one further substance which is selected from the group formed from active ingredients obtainable from plants of the genus Echinacea, and also taurine and derivatives thereof to the skin covered with hair, in particular, scalp, it is possible to modulate the metabolism of these highly specialized cells and to stimulate the ATP synthesis in the hair follicle to a particularly high degree.

Furthermore, it has surprisingly been found that the compositions according to the invention are suitable for stimulating the release of growth factors and for strengthening and thickening the hair by stimulating the proliferation of the hair keratinocytes.

### BRIEF DESCRIPTION OF THE SEVERAL VIEWS OF THE DRAWING(S)

Not Applicable

### DETAILED DESCRIPTION OF THE INVENTION

The present invention relates to cosmetic and pharmaceutical compositions, in particular, for the treatment of the hair or of the skin, comprising at least one compound which is selected from L-carnitine or L-carnitine derivatives, and at least one further substance which is selected from the group formed from active ingredients obtainable from plants of the genus Echinacea, and also taurine and derivatives thereof.

The L-carnitine derivatives are selected, in particular, from acetyl-L-carnitine, L-carnitine fumarate, L-carnitine citrate, lauroyl-L-carnitine and particularly preferably, L-carnitine tartrate. The specified L-carnitine compounds are obtainable, for example, from Lonza GmbH (Wuppertal, Germany).

According to the invention, an active ingredient obtainable from plants of the genus Echinacea is to be understood as meaning the plant itself, its plant parts, extracts and pressed juices of the sun hat plants (Echinacea (synonym: Brauneria NECKER), in particular, from Echinacea angustifolia DC, Echinacea paradoxa (NORTON), Echinacea simulata, E. atrorubens, E. tennesiensis, Echinacea strigosa (MCGREGOR), Echinacea laevigata, Echinacea purpurea (L.) Moench and Echinacea pallida (Nutt), and also active substances to be obtained from these extracts. Particular preference is given to using pressed juices and extracts of Echinacea, in particular, of Echinacea purpurea (L) MOENCH.

Preferably, the pressed juices or extracts are obtained from foliage (the parts of plants above the ground) and/or roots of Echinacea. The pressed juices are preferably obtained by mechanical pressing. Particular preference is given to pressing in accordance with the method patented by Flachsmann as in EP 0 730 830 B1, to the disclosure of which reference is hereby made in its entirety.

The extracts can be produced with water and also polar or nonpolar organic solvents, and mixtures thereof, in the manner known to the person skilled in the art. Extracts which can be obtained by extraction with ethanol or water/ethanol mixtures, and also pressed juice, are preferred.

It is possible to use both extracts either in the original extractant, or extracts/pressed juice in water or other organic solvents and/or mixture thereof, in particular, ethanol and also ethanol/water mixtures. Preference is given to using extracted or pressed material as solid, from which the solvent has been removed (in particular, as gently as possible). However, it is also possible to use those extracts/compressed juices from which the solvent has been partially removed, such that a thickened extract/compressed juice is used. Very particular preference is given to pressed juices from the fresh Echinacea purpurea foliage (Echinacea purpurea Moench herba). In particular, the extracts and/or pressed juices are used in solid form.

According to a particularly preferred embodiment, the active ingredient obtainable from plants of the genus Echinacea is selected from pressed juices and extracts which are obtained from Echinacea purpurea.

According to the invention, derivatives of taurine (2-aminoethanesulfonic acid) are to be understood as meaning, in particular, N-alkyl derivatives of taurine. Of particular suitability are N-monomethyltaurine and N,N-dimethyltaurine,

The compositions according to the invention can preferably comprise L-carnitine or at least one L-carnitine derivative, particularly preferably carnitine tartrate, and also taurine and/or derivatives thereof. Further compositions according to the invention are those which, besides L-carnitine or at least one L-carnitine derivative, particularly preferably carnitine tartrate, comprise an active ingredient obtainable from plants of the genus Echinacea, in particular, from Echinacea purpurea, particularly preferably, pressed juice from the fresh plant.

According to the invention, preference is given in particular, to a combination of L-carnitine or at least one L-carnitine derivative, particularly preferably L-carnitine tartrate, with taurine and/or derivatives thereof and an active ingredient obtainable from plants of the genus Echinacea, in particular, pressed juice from Echinacea purpurea herba.

It was shown that the APT amount in the treated follicles is significantly increased compared to a placebo formulation. The biologically active part of the hair thus has available significantly more ATP as energy supplier for biochemical syntheses and transport processes, meaning that metabolism processes and cellular structures can be optimally maintained and renewed. As a result, the hair becomes stronger and is vitalized and can better repair damage or construct new hair. The increase in the metabolism activity favors hair growth since for the underlying processes, building blocks, such as, for example, amino acids for the construction of protein, have to be adequately provided; the energy required for this is provided, for example, by the metabolism of glucose.

Furthermore, it has been found that the use of the compositions according to the invention leads to stimulation of the hair growth and to an intensification of vital hair. It was possible to demonstrate a synergistic increase in the expression of the growth factors HGF (hepatocyte growth factor) and KGF (keratinocyte growth factor) in dermal papillae cells. The effect of a composition comprising L-carnitine tartrate, taurine and pressed juice from Echinacea purpurea herba is about 13% higher than that which can be achieved by an addition of the individual effects (cf. Example 21).

A particular influence of the compositions according to the invention was established on the biological hair thickening. The stimulation of the keratinocytes of the outer root sheath, which is co-responsible for the formation of the hair shaft, takes place via the growth factors HGF and KGF. Through the hair thickening on a biological basis, effects such as “over-treatment” of the hair are avoided. The hair grows from the root to an increased degree and with a greater diameter, meaning that this effect is particularly long-lasting. This effect was likewise demonstrated in vivo (cf. also Example 26).

A further advantage of the present invention is that the compositions according to the invention are able to have a positive influence on the hair structure by stimulating the special hair-specific structural proteins (the hair keratins). Thus, it was surprisingly found that the gene expression of the hair keratins, hHa3-I and hHa4 is significantly increased. As a result, the hair structure, and thus the hair, becomes strengthened and fortified. By influencing the hair structure in the hair root, the hair can subsequently grow in a strong and healthy manner without secondary phenomena, such as, for example, the accumulation of care substances on the hair fiber.

Furthermore, it has been found that by applying the compositions according to the invention, the hair is positively influenced in its structure, its growth and its metabolism. The gene expression of the hair genes important therefor was significantly regulated through the use of the composition according to the invention. In particular, increased expressions of genes were observed which are important in the extracellular matrix for the anchoring of the hair in the scalp. Both the dermal papillae, and also the entire bulb are surrounded by extracellular matrix which anchors the hair follicle in the scalp. Promoting the synthesis of extracellular matrix proteins such as laminin and collagen leads to a particularly good anchoring of the hair in the scalp.

Surprisingly, the penetration of active ingredients to the follicle is hindered since the corresponding target, the dermal papillae and also the ORS keratinocytes, is embedded in the scalp to a depth of about 2 mm. The use of liposomes increases the penetration of an active ingredient, meaning that liposomal encapsulated compositions according to the invention have a very good effect. Surprisingly, it has been found that the compositions according to the invention even exhibit adequate penetration to the site of action when the use of liposomes is not possible for formulation reasons.

The compositions according to the invention, in particular, for treating the hair or the skin, can comprise L-carnitine and/or an L-carnitine derivative or else a plurality of different L-carnitine derivatives.

L-carnitine or L-carnitine derivatives are present in the compositions according to the invention preferably in amounts of from 0.001 to 10% by weight, based on the total preparation. Amounts of from 0.1 to 10% by weight are particularly preferred, amounts of from 0.1 to 5% by weight are preferred to a particular degree, and amounts of from 1 to 3% by weight are very particularly preferred.

Taurine and/or derivatives thereof are present in the compositions according to the invention preferably in amounts of from 0.001 to 10% by weight, based on the total preparation. Amounts of from 0.05 to 5% by weight are particularly preferred, amounts of from 0.1 to 5% by weight are preferred to a particular degree, and amounts of from 0.5 to 3% by weight are very particularly preferred.

Active ingredients obtainable from plants of the genus Echinacea, preferably the pressed juices and/or extracts of Echinacea, are present in the compositions according to the invention preferably in amounts of from 0.001 to 10% by weight, based on the total preparation. Amounts of from 0.01 to 5% by weight are particularly preferred, amounts of from 0.01 to 5% by weight are preferred to a particular degree, and amounts of from 0.01 to 2% by weight are very particularly preferred.

According to a further preferred embodiment of the present invention, the composition according to the invention can also comprise active ingredients obtainable from plants of the Apiaceae family, in particular, the genus Foeniculum in particular, Feoniculum vulgare (fennel). These may, in particular, be extracts or pressed juices from plant parts or the seed, which can be obtained, for example, in a manner analogous to the extraction methods given above for Echinacea. Preference is given in particular, to fennel seed extract, for example, produced by Flachsmann.

Particular preference is given to compositions according to the invention comprising fennel seed extract, L-carnitine and/or L-carnitine derivatives, preferably, L-carnitine tartrate, and also an extract of Echinacea, preferably, pressed juice from Echinacea purpurea.

The compositions according to the invention can additionally comprise protein hydrolyzates, preferably, cationized protein hydrolyzates, where the underlying protein hydrolyzate can originate from animal, for example, from collagen, milk or keratin, or from vegetable, for example, from wheat, corn, rice, potatoes, soybean or almonds, from marine life forms, for example, from fish collagen or algae, or biotechnically obtained protein hydrolyzates. The protein hydrolyzates on which the cationic derivatives are based can be obtained from the corresponding proteins by a chemical, in particular, alkaline or acidic, hydrolysis, by an enzymatic hydrolysis and/or a combination of both types of hydrolysis. The hydrolysis of proteins usually produces a protein hydrolyzate with a molecular weight distribution of about 100 Daltons to several thousand Daltons. Preference is given to those cationic protein hydrolyzates whose underlying protein fraction has a molecular weight of from 100 to 25,000 Daltons, preferably 250 to 50,000 Daltons. Furthermore, cationic protein hydrolyzates are to be understood as meaning quaternized amino acids and mixtures thereof. The quaternization of the protein hydrolyzates or of the amino acids is often carried out by means of quaternary ammonium salts, such as, for example, N,N-dimethyl-N—(N-alkyl)-N-(2-hydroxy-3-chloro-N-propyl)ammonium halides. Furthermore, the cationic protein hydrolyzates can also be yet further derivatized. Typical examples of the cationic protein hydrolyzates and derivatives that may be mentioned are the products specified under the INCI names in the “International Cosmetic Ingredient Dictionary and Handbook,” (seventh edition 1997, the Cosmetic, Toiletry, and Fragrance Association 1101 17th Street, N.W. Suite 300, Washington, D.C. 20036-4702) and that are commercially available: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

Furthermore, film-forming substances can additionally be incorporated into the formulations; these attach to the hair and thus directly thicken it in a noticeable manner. This can advantageously happen in addition to the effect according to the invention of hair thickening, which takes place by a biological root through stimulation of the hair follicle, meaning that, in addition to the relatively long-lasting thickening of the hair according to the invention through stimulation of the hair follicle, a short-term palpable effect of the composition according to the invention is achieved. Suitable film formers are known to the person skilled in the art and are selected, for example, from polymers, e.g., polyvinyl alcohol or polyvinyl pyrrolidone, and copolymers thereof.

With regard to sequence of events, the use of the compositions according to the invention is not subject to any restrictions of any kind. In principle, it is possible to apply two separate preparations, comprising (a) L-carnitine or an L-carnitine derivative and (b) at least one further component, also the components according to the invention, taurine and/or derivatives thereof, and/or an active ingredient obtainable from plants of the genus Echinacea, to the hair one after the another in any desired order. However, in this connection, very little time should elapse between steps (a) and (b), so that the hair does not dry out between these steps.

The compositions according to the invention can either remain on the hair or, preferably, be rinsed out after a contact time of from 10 seconds to 60 hours. This rinsing can take place with pure water or a standard commercial shampoo. In most cases, contact times of from 1 to 15 minutes have proven adequate.

Irrespective of the exact course of the treatment, it has proven advantageous to use the compositions according to the invention at a temperature of from 20 to 55° C., in particular, from 35 to 40° C.

As regards the nature according to which compositions according to the invention are applied to the hair, there are, in principle, no limitations.

Of particular interest according to the invention are hair tonics, in particular, as a leave on formulation. These are preferably used at room temperature. The alcoholic content is preferably in the range from about 30% to about 35%. The pH should be about pH 7. Particularly in the case of hair tonics, the use of L-carnitine or L-carnitine derivatives encapsulated in liposomes has proven advantageous.

Here, it is possible to begin by mixing the components according to the invention, in particular, the mixture of L-carnitine tartrate, an active ingredient obtainable from plants of the genus Echinacea, particularly preferably pressed juice from Echinacea purpurea, and taurine, and then to encapsulate the mixture. However, it is also possible to individually encapsulate the respective components and then to use them in the corresponding amount in the composition according to the invention.

The mixing of the components, in particular, of the mixture of L-carnitine tartrate, an active ingredient obtainable from plants of the genus Echinacea, particularly preferably pressed juice from Echinacea purpurea, and taurine, with subsequent encapsulation in liposomes is particularly preferred. Such liposomes can be used in particular, in hair tonics.

Although the above-mentioned hair treatment compositions are preferred according to the invention, the combinations according to the invention, in particular, the compositions comprising L-carnitine tartrate, taurine and an active ingredient obtainable from plants of the genus Echinacea, particularly preferably pressed juice from Echinacea purpurea, can also be added to other hair treatment compositions, such as, for example, hair colorants and waving compositions. These compositions optionally comprise the known direct dyes, precursors of oxidation dyes (developer and coupler components) and oxidizing agents or reducing agents.

Advantageously, the compositions according to the invention can thus protect the hair against stress during coloring, activate the hair follicle and at the same time reduce and/or alleviate skin irritations caused by the waving compositions or hair colorants.

The compositions according to the invention are likewise suitable for the treatment of skin. In the context of the invention, the term “skin” is to be understood as meaning in particular, human skin and mucous membrane.

The compositions according to the invention likewise bring about the thickening of epithelial cells and cell layers, in particular, on the skin, an improvement in the strength of the skin, the fortification of the epidermis, a lessening in thinning of the skin, in particular, through aging phenomena of the skin, a reduction in the transepidermal water loss of the skin, an improvement in skin moisture, the protection of the skin against infections, exogenous factors such as smog, cigarette smoke and against stress through harmful and/or irritative substance, in particular, surfactants and/or frequent water contact.

Suitable as formulation of the preparations comprising the compositions according to the invention are, for example, creams, lotions, solutions, tonics, emulsions such as W/O, O/W, PIT emulsions (emulsions according to the teaching of phase inversion, called PIT), microemulsions and multiple emulsions, gels, sprays, aerosols and foam aerosols. These are generally formulated on an aqueous or aqueous-alcoholic basis. As alcoholic component, use is made here of lower alkanols and also polyols, such as propylene glycol and glycerol. Ethanol and isopropanol are preferred alcohols. Water and alcohol may be present in the aqueous alcoholic base in a weight ratio of from 1:10 to 10:1. Tonics and aqueous-alcoholic mixtures which comprise up to 50% by weight, in particular, up to 25% by weight, of alcohol, based on the alcohol/water mixture may be bases preferred according to the invention. The pH of these preparations can in principle be at values of 2-11. It is preferably between 2 and 7, with values from 3 to 5 being particularly preferred. For adjusting this pH, virtually every acid or base that can be used for cosmetic purposes can be used. Usually, food acids are used as acids. Food acids are understood as meaning those acids which are consumed in the course of usual food consumption and have positive effects on the human organism. Food acids are, for example, acetic acid, lactic acid, tartaric acid, citric acid, malic acid, ascorbic acid and gluconic acid. For the purposes of the invention, the use of citric acid and lactic acid is particularly preferred. Preferred bases are ammonia, alkali metal hydroxides, triethanolamine and N,N,N′,N′-tetrakis(2-hydroxypropyl)ethylenediamine.

The compositions can be formulated as single-chamber system or as twin-chamber system.

Besides the active ingredients obligatorily required according to the invention, the compositions can, in principle, comprise all further components known to the person skilled in the art for such cosmetic compositions.

According to a particularly preferred embodiment, the compositions according to the invention comprises a hair-growth-stimulating active ingredient. As hair-growth-stimulating active ingredients, particular preference is given to using those compounds which are selected from 5-α-reductase inhibitors, minoxidil (6-piperidino-2,4-pyrimidinediamine 3-oxide) and aminexil (diaminopyridimine oxide).

5-α-reductase inhibitors are in particular, functional C₂-C₁₂-carboxylic acids and physiologically compatible metal salts thereof, in particular, 10-hydroxydecanoic acid, 10-hydroxydecenoic acid and its derivatives, derivatives of C₃-C₉-polyols, phenol derivatives, plant extracts, fragrances, flavonoids, isoflavonoids, 6,7-disubstituted 2,2-dialkylchromanes or -chromenes, aluminum chlorohydrate, 2-phenylethanol, etidronic acid, 7-acetyl-1,1,3,4,4,6-hexamethyltetraline, tropolone derivatives, esters of sulfuric acid with alkoxylated C₈-C₁₈-fatty alcohols and physiologically compatible metal salts thereof, esters of phosphoric acid and of triphosphoric acid with mono- to hexavalent hydroxy compounds, silicic acid esters, mycosporin-like amino acids (MAA) which can be isolated from marine organisms, and quaternary silicone compounds.

Derivatives are to be understood as meaning, in particular, salts, esters and amides thereof.

Very particular preference here is given to 10-hydroxydecanoic acid, 10-hydroxydecenoic acid and finasteride (N-tert-butyl-3-oxo-4-aza-5α-androst-1-ene-17β-carboxamide) and derivatives thereof.

It has been found that the hair-growth-stimulating effect of the active ingredients can be further improved through their use in compositions according to the invention. Particular preference is given to compositions comprising L-carnitine tartrate, an active ingredient obtainable from plants of the genus Echinacea, particularly preferably, pressed juice from Echinacea purpurea, taurine and at least one active ingredient selected from 10-hydroxydecanoic acid, 10-hydroxydecenoic acid, minoxidil and finasteride. The hair-growth-stimulating active ingredient is very particularly preferably also selected from minoxidil and finasteride in this combination.

Further active ingredients, auxiliaries and additives are, for example:
nonionogenic surfactants, such as, for example, alkylphenol polyglycol ethers, fatty acid polyglycol esters, fatty acid amide polyglycol ethers, fatty amine polyglycol ethers, alkoxylated triglycerides, such as, in particular, ethoxylated castor oil, alk(en)yl oligoglucosides, fatty acid N-alkylglucamides, polyol fatty acid esters, sugar esters, sorbitan esters and polysorbates. If the nonionic surfactants comprise polyglycol ether chains, they can have a conventional or narrowed homolog distribution.
anionic surfactants, in particular, alkyl sulfates, alkyl polyglycol ether sulfates and ether carboxylic acids having 10 to 18 carbon atoms in the alkyl group and up to 12 glycol ether groups in the molecule, soaps, and sulfosuccinic acid mono- and dialkyl esters having 8 to 18 carbon atoms in the alkyl group and sulfosuccinic acid monoalkylpolyoxyethyl esters having 8 to 18 carbon atoms in the alkyl group and 1 to 6 oxyethyl groups,
zwitterionic surfactants, in particular, the betaines, such as the N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, and 2-alkyl-3-carboxyl-methyl-3-hydroxyethylimidazolines having in each case 8 to 18 carbon atoms in the alkyl or acyl group, and cocoacylaminoethylhydroxyethyl carboxymethyl glycinate,
ampholytic surfactants, such as, for example, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group,
nonionic polymers, such as, for example, vinylpyrrolidone/vinyl acrylate copolymers, polyvinylpyrrolidone and vinylpyrrolidone/vinyl acetate copolymers and polysiloxanes,
thickeners, such as agar agar, guar gum, alginates, xanthan gum, gum Arabic, karaya gum, carob seed flour, linseed gums, dextrans, cellulose derivatives, e.g., methylcellulose, hydroxyalkylcellulose and carboxymethylcellulose, starch fractions and derivatives, such as amylose, amylopectin and dextrins, clays, such as, for example, bentonite or completely synthetic hydrocolloids, such as, for example, polyvinyl alcohol,
structurants, such as maleic acid and lactic acid,
hair-conditioning compounds, such as phospholipids, for example, soybean lecithin, egg lecithin and cephalins, and also silicone oils,
perfume oils, dimethyl isosorbide and cyclodextrins,
solvents and solubility promoters, such as ethanol, isopropanol, ethylene glycol, propylene glycol, glycerol and diethylene glycol,
symmetrical and asymmetrical, linear and branched dialkyl ethers having in total between 12 and 36 carbon atoms, in particular, 12 to 24 carbon atoms, such as, for example, di-n-octyl ether, di-n-decyl ether, di-n-nonyl ether, di-n-undecyl ether and di-n-dodecyl ether, n-hexyl n-octyl ether, n-octyl n-decyl ether, n-decyl n-undecyl ether, n-undecyl n-dodecyl ether and n-hexyl n-undecyl ether, and di-tert-butyl ether, diisopentyl ether, di-3-ethyldecyl ether, tert-butyl n-octyl ether, isopentyl n-octyl ether and 2-methylpentyl n-octyl ether,
fatty alcohols, in particular, linear and/or saturated fatty alcohols having 8 to 30 carbon atoms, and monoesters of fatty acids with alcohols having 6 to 24 carbon atoms,
active ingredients that improve fiber structure, in particular, mono-, di- and oligosaccharides, such as, for example, glucose, galactose, fructose, fruit sugars and lactose,
conditioning active ingredients, such as paraffin oils, vegetable oils, e.g., sunflower oil, orange oil, almond oil, wheat germ oil and peach kernel oil, and also phospholipids, for example, soybean lecithin, egg lecithin and cephalins,
quaternized amines, such as methyl-1-alkylamidoethyl-2-alkylimidazolinium methosulfate,
antifoams, such as silicones,
dyes for coloring the composition,
antidandruff active ingredients, such as piroctone olamine, zinc omadine and climbazole,
photo protective agents, in particular, derivatized benzophenones, cinnamic acid derivatives and triazines,
further substances for adjusting the pH, such as, for example, α- and β-hydroxycarboxylic acids
active ingredients, such as allantoin and bisabolol,
cholesterol,
consistency regulators, such as sugar esters, polyol esters or polyol alkyl ethers,
fats and waxes, such as spermaceti, beeswax, montan wax and paraffins,
fatty acid alkanolamides,
complexing agents, such as EDTA, NTA, β-alaninediacetic acid and phosphonic acids,
swelling and penetration substances, such as glycerol, propylene glycol monoethyl ether, carbonates, hydrogen carbonates, guanidines, ureas, and primary, secondary and tertiary phosphates,
opacifiers, such as latex, styrene/PVP copolymers and styrene/acrylamide copolymers
pearlizing agents, such as ethylene glycol mono- and distearate, and PEG-3-distearate,
pigments,
reducing agents, such as, for example, thioglycolic acid and derivatives thereof, thiolactic acid, cysteamine, thiomalic acid and α-mercaptoethanesulfonic acid,
propellants, such as propane-butane mixtures, N₂O, dimethyl ether, CO₂ and air,
antioxidants.

Furthermore, the compositions according to the invention can comprise surfactants. These may either be anionic, ampholytic, zwitterionic or nonionogenic surfactants, or else cationic surfactants.

In a preferred embodiment of the present invention, a combination of anionic and nonionic surfactants or a combination of anionic and amphoteric surfactants is used, for example, in a shampoo. However, in a hair tonic, the person skilled in the art can also largely or completely dispense with the use of surfactants.

In individual cases, it has proven advantageous to select the surfactants from amphoteric or nonionic surfactants.

Suitable anionic surfactants in compositions according to the invention are all anionic surface-active substances suitable for use on the human body. These are characterized by a water-solubilizing, anionic group, such as, for example, a carboxylate, sulfate, sulfonate or phosphate group and a lipophilic alkyl group having about 10 to 22 carbon atoms. Additionally, glycol or polyglycol ether groups, ester, ether and amide groups and also hydroxyl groups may be present in the molecule.

Nonionogenic surfactants comprise, as hydrophilic group, e.g., a polyol group, a polyalkylene glycol ether group or a combination of a polyol and polyglycol ether group. Such compounds are, for example:
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂-fatty acid mono- and diesters of addition products of from 1 to 30 mol of ethylene oxide onto glycerol,
C₈-C₂₂-alkyl mono- and oligoglycosides and ethoxylated analogs thereof, and
addition products of from 5 to 60 mol of ethylene oxide onto castor oil and hydrogenated castor oil.

Preferred nonionic surfactants are alkyl polyglycosides of the general formula R¹O-(Z)ₓ. These compounds are characterized by the following parameters.

The alkyl radical R¹ comprises 6 to 22 carbon atoms and may either be linear or branched. Preference is given to primary linear and 2-methyl-branched aliphatic radicals. Such alkyl radicals are, for example, 1-octyl, 1-decyl, 1-lauroyl, 1-myristyl, 1-cetyl and 1-stearyl. Particular preference is given to 1-octyl, 1-decyl, 1-lauroyl, 1-myristyl. When using “oxo alcohols” as starting materials, compounds with an uneven number of carbon atoms in the alkyl chain predominate.

The alkyl polyglycosides that can be used according to the invention can comprise, for example, only one specific alkyl radical R¹. Usually, however, these compounds are produced starting from natural fats and oils or mineral oils. In this case, the alkyl radicals R present are mixtures corresponding to the starting compounds or corresponding to the particular work-up of these compounds.

Particular preference is given to those alkyl polyglycosides in which R¹ consists
essentially of C₈- and C₁₀-alkyl groups,
essentially of C₁₂- and C₁₄-alkyl groups,
essentially of C₈- to C₁₆-alkyl groups or
essentially of C₁₂- to C₁₆-alkyl groups.

Any mono- or oligosaccharides can be used as sugar building block Z. Usually, sugars having 5 or 6 carbon atoms, and the corresponding oligosaccharides are used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides that can be used according to the invention comprise, on average, 1.1 to 5 sugar units. Alkyl polyglycosides with x values of from 1.1 to 1.6 are preferred. Very particular preference is given to alkyl glycosides in which x is 1.1 to 1.4.

Besides their surfactant effect, the alkyl glycosides can also serve to improve the fixing of fragrance components on the hair. Thus, whenever an effect of the perfume oil on the hair that extends beyond the duration of the hair treatment is desired, the person skilled in the art will preferably have recourse to this substance class as further ingredient of the preparations according to the invention.

The alkoxylated homologs of the specified alkyl polyglycosides can also be used according to the invention. These homologs can comprise, on average, up to 10 ethylene oxide and/or propylene oxide units per alkyl glycoside unit.

Furthermore, it is also possible to use zwitterionic surfactants, in particular, as cosurfactants. “Zwitterionic surfactants” is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the betaines, such as the N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, and 2-alkyl-3-carboxylmethyl-3-hydroxyethylimidazolines having in each case 8 to 18 carbon atoms in the alkyl or acyl group, and also cocoacylaminoethylhydroxyethyl carboxymethyl glycinate. A preferred zwitterionic surfactant is the fatty acid amide derivative known under the INCI name Cocamidopropyl Betaine.

Ampholytic surfactants are likewise suitable in particular, as cosurfactants. Ampholytic surfactants are understood as meaning those surface-active compounds which, apart from a C₈-C₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂₋₁₈-acylsarcosine.

According to the invention, the cationic surfactants used are in particular, those of the quaternary ammonium compound type, of the ester quat type and of the amidoamine type.

Preferred quaternary ammonium compounds are ammonium halides, in particular, chlorides and bromides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauroyldimethylammonium chloride, lauroyldimethylbenzylammonium chloride and tricetylmethylammonium chloride, and also the imidazolium compounds known under the INCI names Quaternium-27 and Quaternium-83. The long alkyl chains of the above-mentioned surfactants preferably have 10 to 18 carbon atoms.

Ester quats are known substances which contain both at least one ester function and also at least one quaternary ammonium group as structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trade names Stepantex®, Dehyquart® and Armocare®. The products Armocare® VGH-70, an N,N-bis(2-palmitoyloxyethyl)dimethylammonium chloride, and Dehyquart® F-75 and Dehyquart® AU-35 are examples of such ester quats.

The alkylamidoamines are usually produced by amidation of natural or synthetic fatty acids and fatty acid cuts with dialkylaminoamines. One compound from this substance group which is particularly suitable according to the invention is the stearamidopropyldimethylamine commercially available under the name Tegoamid® S 18.

The compounds with alkyl groups used as surfactant may each be single substances. However, it is generally preferred, for the production of these substances, to start from native vegetable or animal raw materials, giving substance mixtures with varying alkyl chain lengths depending on the particular raw material.

In the case of the surfactants which are addition products of ethylene oxide and/or propylene oxide onto fatty alcohols or derivatives of these addition products, it is possible to use either products with a “normal” homolog distribution, or those with a narrowed homolog distribution. Here, “normal” homolog distribution is understood as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ether carboxylic acids, alkaline earth metal oxides, hydroxides or alkoxides are used as catalysts. The use of products with a narrowed homolog distribution may be preferred.

With regard to further optional components and the amounts of these compounds used, reference is made expressly to the relevant handbooks known to the person skilled in the art, e.g., Kh. Schrader, Grundlagen und Rezepturen der Kosmetika [Fundamentals and Formulations of Cosmetics], 2nd edition, Hüthig Buch Verlag, Heidelberg, 1989.

The present invention further provides the use, in particular, the cosmetic use for the vitalization of hair, stimulation of the energy metabolism in hair follicles, activation of hair follicles, promotion or intensification of hair growth, hair thickening, treatment of hair loss and for influencing (in particular, stimulating) keratin synthesis, or for conditioning hair.

Particular preference is given to the use, in particular, the cosmetic use, of a composition comprising L-carnitine and/or at least one L-carnitine derivative which is selected from acetyl-L-carnitine, L-carnitine fumarate, L-carnitine citrate, lauroyl-L-carnitine and in particular, L-carnitine tartrate, in combination with taurine and/or derivatives thereof, and/or an active ingredient obtainable from plants of the genus Echinacea.

Very particular preference is given to the use of a composition comprising L-carnitine tartrate, pressed juice from Echinacea purpurea herb. and taurine and/or derivatives thereof.

The present invention further provides the use, in particular, the cosmetic use, for the thickening of eptheliumial cells and cell layers, in particular, on the skin, improving the strength of the skin, boosting the epidermis, lessening the thinning of the skin, in particular, combating aging symptoms of the skin, reducing the transepidermal water loss of the skin, improving the skin moisture, protecting the skin against infections, exogenous factors such as smog, cigarette smoke and also against stress through surfactants and/or frequent water contact.

Very particular preference is given to the use of a composition comprising L-carnitine tartrate, an active ingredient obtainable from plants of the genus Echinacea, particularly preferably pressed juice from Echinacea purpurea, and taurine and/or derivatives thereof, in particular, taurine.

The present invention further provides a method, in particular, a cosmetic method, for the vitalization of hair, hair thickening, stimulation of keratin synthesis, stimulation of the energy metabolism in hair follicles, activation of hair follicles, promotion or intensification of hair growth or for hair conditioning, characterized in that a composition according to the invention, in particular, in the combination of L-carnitine and/or derivatives thereof, taurine and/or derivatives thereof and an active ingredient obtainable from plants of the genus Echinacea, is applied to the hair or the skin covered with hair.

The present invention further provides a method, in particular, a cosmetic method, for thickening eptheliumial cells and cell layers, in particular, on the skin, improving the strength of the skin, for boosting the epidermis, lessening the thinning of the skin, in particular, of the skin, reducing the transepidermal water loss of the skin, improving the skin moisture, protecting the skin against infections, exogenous factors such as smog, cigarette smoke, and against the stress through harmful and/or irritative substance, in particular, surfactants and/or frequent water contact, characterized in that a composition according to the invention, in particular, in the combination of L-carnitine and/or derivatives thereof, taurine and/or derivatives thereof and an active ingredient obtainable from plants of the genus Echinacea, is applied to the skin.

Very particularly preferably, such a method is carried out using a composition comprising L-carnitine tartrate, taurine and pressed juice from Echinacea purpurea herb.

The examples which follow are intended to elucidate the subject matter of the invention in more detail without imposing any restriction on it.

All data are in percent by weight (wt. %).

Information on the substances used in the examples:

### Pressed juice from Echinacea purpurea.

### Pressed juice from Echinacea purpurea (L.) Moench, Echinacea purp. hba. succ. sicc, EFLA894, Flachsmann (article No. 008594), produced according to the method patented by Flachsmann (EP-B-0 730 830), solid

### Gluadin WQ.

### Cognis Deutschland GmbH,

### AQUA (WATER), LAURDIMONIUM HYDROXYPROPYL HYDROLYZED WHEAT PROTEIN, ETHYLPARABEN, METHYLPARABEN

### Protein hydrolyzates, wheat germ, (3-(dodecyldi methylammonio)-2-hydroxypropyl), chlorides about 30-35% content

### Gluadin WLM.

### Cognis Deutschland GmbH

### Wheat protein hydrolyzate in H2O

### INCI declaration [INCI] HYDROLYZED WHEAT PROTEIN

### Content about 20-24%

### Salcare SC 96.

### Ciba

### INCI declaration [INCI] POLYQUATERNIUM-37, PROPYLENE GLYCOL DICAPRYLATE/DICAPRATE

### Content about 50%

### Cetiol HE.

### Cognis Deutschland GmbH

### Cocomonoglyceride ethoxylated (7 EO)

### INCI declaration [INCI] PEG-7 GLYCERYL COCOATE

### Sepigel 305.

### Seppic (Interorgana)

### INCI declaration [INCI] POLYACRYLAMIDE, C13-14 ISOPARAFFIN, LAURETH-7

### Content about 45-50%

### Euperlan PK 3000.

### Cognis Deutschland GmbH

### INCI declaration [INCI] GLYCOL DISTEARATE, GLYCERIN, LAURETH-4, COCAMIDOPROPYL BETAINE

### Plantacare 818 UP.

### Cognis Deutschland GmbH

### C8-16 alkyl polyglucoside

### *NLP

### COCO-GLUCOSIDE, AQUA (WATER)

### Ajidew NL 50.

### Ajinomoto

### Pyrrolidonecarboxylic acid sodium salt

### Na PCA

### SODIUM PCA

### Uvinul MS 40.

### BASF

### Hydroxy-4-methoxybenzophenone-5-sulfonic acid *2-BENZOPHENONE-4

### Arlypon F.

### Cognis Deutschland GmbH

### Lauromacrogol JP 12 (Pharmacopoe of Japan)

### *NLP

### C12-14 fatty alcohols ethoxylated (2.5 EO)

### LAURETH-2

### Antil 171.

### Goldschmidt (Degussa)

### Polyol fatty acid ester

### PEG-18 GLYCERYL OLEATE/COCOATE

### Synthalen K.

### Synthalen KD (formerly)

### 3V Sigma

### Polyacrylic acid

### CARBOMER

### Cremophor RH 40.

### BASF

### Fragrance C 041

### Castor oil, hydrogenated, ethoxylated (45 EO)

### PEG-40 HYDROGENATED CASTOR OIL

### Neutrol TE.

### BASF

### Tetrakis(2-hydroxypropyl)ethylenediamine *N,N,N′,N′-edetol

### TETRAHYDROXYPROPYL ETHYLENEDIAMINE

### EXAMPLE 1

### Hair Rinse

| | |
|---|---|
| L-carnitine | 2.0 |
| Taurine | 1.0 |
| Echinacea purpurea pressed juice (Flachsmann) | 0.1 |
| Eumulgin ® B2¹ | 0.3 |
| Cetyl/stearyl alcohol | 3.3 |
| Isopropyl myristate | 0.5 |
| Paraffin oil perliquidum 15 cSt. DAB 9 | 0.3 |
| Dehyquart ® A-CA² | 2.0 |
| Gluadin WQ | 0.2 |
| Gluadin WLM | 0.5 |
| Pantolactone | 0.5 |
| Subtilisin or papain | 0.5 |
| Phenonip ®³ | 0.8 |
| Water | ad 100 |

pH = 7.0
¹Cetylstearyl alcohol + 20 EO (INCI name: Ceteareth-20) (HENKEL)
²Trimethylhexadecylammonium chloride (about 25% active substance in water; INCI name: Aqua, Cetrimonium Chloride)
³Methyl hydroxybenzoate/ethyl hydroxybenzoate/propyl hydroxybenzoate/butyl hydroxybenzoate/phenoxyethanol mixture (about 28% active substance; INCI name: Phenoxyethanol, Methylparaben, Ethylparaben, Propylparaben, Butylparaben) (NIPA)

### EXAMPLE 2

### Hair Rinse

| | |
|---|---|
| Acetyl-L-carnitine | 1.0 |
| Echinacea purpurea pressed juice (Flachsmann) | 0.1 |
| Taurine | 0.5 |
| Eumulgin ® B2 | 0.3 |
| Cetyl/stearyl alcohol | 3.3 |
| Isopropyl myristate | 0.5 |
| Paraffin oil perliquidum 15 cSt. DAB 9 | 0.3 |
| Dehyquart ® L 80⁵ | 2.0 |
| Gluadin WQ | 0.5 |
| Gluadin WLM | 0.2 |
| Pantolactone | 1.0 |
| Subtilisin or papain | 1.0 |
| Citric acid | 0.4 |
| Phenonip ® | 0.8 |
| Water | ad 100 |

pH = 7.0
⁵Bis(cocoylethyl)hydroxyethylmethylammonium methosulfate (about 76% active substance in propylene glycol; INCI name: Dicocoylethyl Hydroxyethylmonium Methosulfate, Propylene Glycol) (HENKEL)

### EXAMPLE 3

### Hair Rinse

| | |
|---|---|
| L-carnitine tartrate | 2.0 |
| Echinacea purpurea pressed juice (Flachsmann) | 0.5 |
| Taurine | 2 |
| Isopropyl myristate | 0.50 |
| Paraffinum liquidum | 0.50 |
| Cetearyl alcohol | 2.5 |
| Eumulgin B 2* | 0.40 |
| Citric acid | 0.20 |
| Propylparaben | 0.20 |
| Water, demineralized | ad 100 |
| Phenoxyethanol, pure | 0.30 |
| Methylparaben | 0.20 |
| Dehyquart ® F 75 | 2.0 |

*Eumulgin B 2 = Ceteareth-20

### EXAMPLE 4

### Hair Treatment (Rinse Off)

| | |
|---|---|
| L-carnitine fumarate | 4.0 |
| N-monomethyl taurine | 1 |
| Echinacea purpurea pressed juice (Flachsmann) | 0.25 |
| Dehyquart ® F75⁷ | 4.0 |
| Cetyl/stearyl alcohol | 4.0 |
| Paraffin oil perliquidum 15 cSt. DAB 9 | 1.5 |
| Dehyquart ® A-CA | 4.0 |
| Salcare ® SC 96 | 0.5 |
| Gluadin WQ | 1.0 |
| Gluadin WLM | 1.0 |
| Pantolactone | 0.5 |
| Subtilisin or Papain | 0.2 |
| Citric acid | 0.15 |
| Phenonip ® | 0.8 |
| Water | ad 100 |

pH = 7.0
⁷fatty alcohols/methyltriethanolammonium methyl sulfate dialkyl ester mixture (INCI name: Distearoylethyl Hydroxyethylmonium Methosulfate, Cetearyl Alcohol) (Cognis)

### EXAMPLE 5

### Hair Treatment (Rinse Off)

| | |
|---|---|
| L-carnitine citrate | 2.0 |
| Extract from Echinacea angustifolia | 0.2 |
| Taurine | 1.5 |
| Dehyquart ® L 80 | 4.0 |
| Cetyl/stearyl alcohol | 6.0 |
| Paraffin oil perliquidum 15 cSt. DAB 9 | 2.0 |
| Rewoquat ® W 75⁹ | 2.0 |
| Sepigel ® 305 | 0.5 |
| Gluadin WQ | 0.2 |
| Gluadin WLM | 0.5 |
| Pantolactone | 0.5 |
| Subtilisin or Papain | 0.5 |
| Citric acid | 0.15 |
| Phenonip ® | 0.8 |
| Water | ad 100 |

pH = 7.0
⁹1-methyl-2-nortallow-alkyl-3-tallow-fatty acid amidoethylimidazolinium methosulfate (about 75% active substance in propylene glycol; INCI name: Quaternium-27, Propylene Glycol) (WITCO)

### EXAMPLE 6

### Hair Treatment (Leave In)

| | |
|---|---|
| Lauroyl-L-carnitine | 3.0 |
| Echinacea purpurea pressed juice (Flachsmann) | 0.2 |
| Taurine | 1.7 |
| Dehyquart ® F 75 | 0.3 |
| Salcare ® SC 96 | 5.0 |
| Dow Corning ® 200 Fluid, 5 cSt.¹⁰ | 1.5 |
| Gafquat ® 755N¹¹ | 1.5 |
| Biodocarb ®¹² | 0.8 |
| Gluadin WQ | 0.2 |
| Gluadin WLM | 0.5 |
| Pantolactone | 0.5 |
| Subtilisin or Papain | 0.5 |
| Perfume oil | 0.25 |
| Water | ad 100 |

pH = 7.0
¹⁰Polydimethylsiloxane (INCI name: Dimethicone) (DOW CORNING)
¹¹Dimethylaminoethyl methacrylate-vinylpyrrolidone copolymer, quaternized with diethyl sulfate (19% active substance in water; INCI name: Polyquaternium-11) (GAF)
¹²3-Iodo-2-propynyl n-butylcarbamate (INCI name: Iodopropynyl Butylcarbamate) (MILKER & GRUNING)

### EXAMPLE 7

### Hair Treatment (Leave In)

| | |
|---|---|
| L-carnitine tartrate | 3.0 |
| Taurine | 0.9 |
| Echinacea purpurea, ethanolic extract, solid | 0.2 |
| Sepigel ® 305 | 5.0 |
| Dow Corning ® Q2-5220 5 cSt.¹³ | 1.5 |
| Genamin ® DSAC¹⁴ | 0.3 |
| Phenonip ® | 0.8 |
| Gluadin WQ | 0.5 |
| Gluadin WLM | 0.8 |
| Pantolactone | 1.0 |
| Subtilisin or Papain | 0.8 |
| Perfume oil | 0.25 |
| Water | ad 100 |

pH = 7.0
¹³Silicone-glycol copolymer (INCI name: Dimethicone Copolyol) (DOW CORNING)
¹⁴Dimethyldistearylammonium chloride (INCI name: Distearyldimonium Chloride) (CLARIANT)

### EXAMPLE 8

### Hair Treatment

| | |
|---|---|
| L-carnitine tartrate | 2.0 |
| N,N-dimethyl taurine | 1.0 |
| Echinacea purpurea pressed juice (Flachsmann) | 0.2 |
| Cetearyl alcohol | 5.00 |
| Propylparaben | 0.20 |
| Stearamidopropyldimethylamine | 1.50 |
| Dehyquart F 75¹⁵ | 1.50 |
| Paraffinum liquidum | 1.00 |
| Quaternium-87 in propylene glycol | 1.50 |
| Isopropyl myristate | 2.00 |
| Cutina GMS¹⁶ | 1.00 |
| Methylparaben | 0.20 |
| Water | ad 100 |
| Citric acid | 0.45 |
| Dehyquart A CA | 5.00 |
| Rheocare CTH (E) | 0.50 |
| Polymer JR 400 | 0.20 |
| Pantolactone | 0.20 |
| Nicotinamide | 0.10 |
| Phenoxyethanol, pure | 0.40 |
| D-panthenol 75% | 0.20 |
| Dow Corning 1403 Fluid | 1.50 |
| Perfume | 0.20 |

¹⁵Dehyquart F 75 = Distearoylethyl Hydroxyethylmonium Methosulfate
¹⁶Cutina GMS = Glyceryl Monostearate

### EXAMPLE 9

### Shampoo

| | |
|---|---|
| L-carnitine tartrate | 1.5 |
| Taurine | 0.5 |
| Echinacea purpurea pressed juice (Flachsmann) | 0.05 |
| LAURETH SULFATE 25% | 40 |
| CITRIC ACID | 0.1 |
| SODIUM BENZOATE | 0.5 |
| DISODIUM COCOAMPHODIACETATE | 6.0 |
| SALICYLIC ACID | 0.1 |
| HYDROTRITICUM WQ | 1.0 |
| Gluadin WQ | 0.2 |
| Gluadin WLM | 0.5 |
| Pantolactone | 0.5 |
| Subtilisin or papain | 0.2 |
| CETIOL HE | 0.5 |
| PERFUME | 0.4 |
| NACL | 0.5 |
| WATER | ad 100 |

### EXAMPLE 10

### Shampoo

| | |
|---|---|
| L-carnitine citrate | 2.0 |
| Extract from Echinacea pallida | 0.2 |
| N,N-diethyl taurine | 1.3 |
| LAURETH SULFATE 25% (ALKALINE DILUTION) | 25.0 |
| CITRIC ACID MONO REGULAR | 0.3 |
| TIMIRON | 0.5 |
| SODIUM BENZOATE | 0.5 |
| PANTHENOL 75 L | 0.2 |
| EUPERLAN PK 3000 | 8.0 |
| PLANTACARE 818 UP | 2.0 |
| UVINUL MS40 | 1.0 |
| SALICYLIC ACID | 0.2 |
| AJIDEW NL-50 | 2.0 |
| CUTINA HR GROUND | 0.5 |
| CETIOL HE | 1.0 |
| CITRIC ACID MONO REGULAR | 0.03 |
| JAGUAR EXCEL | 0.3 |
| Gluadin WQ | 0.2 |
| Gluadin WLM | 0.5 |
| Pantolactone | 0.5 |
| Subtilisin or papain | 0.5 |
| SODIUM CHLORIDE | 0.3 |
| WATER | ad 100 |

### EXAMPLE 11

### Shampoo

| | |
|---|---|
| L-carnitine | 2.0 |
| Echinacea purpurea pressed juice (Flachsmann) | 0.5 |
| TAURINE | 1 |
| LAURETH SULFATE 25% (ALKALINE DILUTION) | 50 |
| CITRIC ACID MONO REGULAR | 0.4 |
| ARLYPON F | 0.5 |
| ANTIL 171 | 0.3 |
| WHEAT PROTEIN HYDROLYZATE CATIONIZED | 1.5 |
| SODIUM BENZOATE | 0.5 |
| EUPERLAN PK 3000 | 6.0 |
| COCAMIDOPROPYL BETAINE 45% | 5.0 |
| SALICYLIC ACID | 0.2 |
| SILSOFT A-858 | 0.3 |
| Gluadin WQ | 1.0 |
| Glaudin WLM | 1.0 |
| Pantolactone | 0.5 |
| Subtilisin or papain | 0.2 |
| CUTINA HR | 0.2 |
| CETIOL HE | 1.0 |
| WATER | ad 100 |

### EXAMPLE 12

### Shampoo

| | |
|---|---|
| L-carnitine tartrate | 1.0 |
| Echinacea purpurea pressed juice (Flachsmann) | 0.05 |
| Taurine | 1.0 |
| Precursor Laureth sulf. 25% alkaline dilution | 40.00 |
| Citric acid | 0.15 |
| Disodium cocoamphodiacetate | 7.00 |
| Na benzoate | 0.50 |
| Salicylic acid | 0.20 |
| Perfume | 0.15 |
| Sodium chloride | 1.50 |
| Water, demineralized | ad 100 |
| Polymer JR 400 | 0.30 |

### EXAMPLE 13

### Hair Styling Gel

| | |
|---|---|
| Acetyl-L-carnitine | 2.5 |
| Echinacea purpurea pressed juice (Flachsmann) | 0.5 |
| Taurine | 1 |
| SYNTHALEN K | 0.6 |
| NEUTROL TE | 0.5 |
| GLYCEROL DAB 9, 86.5 | 8.00 |
| ETHYL ALCOHOL DENATURED 96 VOL % FLS | 30.00 |
| Gluadin WQ | 0.5 |
| Gluadin WLM | 0.5 |
| Pantolactone | 1.0 |
| Subtilisin or papain | 0.2 |
| POLYETHYLENE GLYCOL | 2.00 |
| PVP/VA W-635 | 6.50 |
| CREMOPHOR RH 40 | 1.00 |
| PERFUME | 0.50 |
| DEIONIZED WATER | ad 100 |

pH = 7.0

### EXAMPLE 14

### Hair Spray

| | |
|---|---|
| L-carnitine tartrate | 2.0 |
| Taurine | 0.8 |
| Echinacea purpurea pressed juice (Flachsmann) | 0.1 |
| AMPHOMER | 3.00 |
| LUVISKOL VA 37 | 16.00 |
| AMP AMINOMETHYLPROPANOL 100 | 0.60 |
| ISOPROPYL MYRISTATE | 0.12 |
| Gluadin WQ | 0.5 |
| Gluadin WLM | 0.5 |
| Pantolactone | 0.2 |
| Subtilisin or papain | 1.0 |
| PERFUME | 0.20 |
| DEIONIZED WATER | ad 100 |
| ETHYL ALCOHOL DENATURED 96 VOL % FLS | 67.50 |

pH = 7.0

### EXAMPLE 15

### Hair Tonic

| | |
|---|---|
| Lauroyl-L-carnitine | 1.0 |
| Echinacea purpurea pressed juice (Flachsmann) | 0.05 |
| Taurine | 1 |
| DEIONIZED WATER | ad 100 |
| PANTHENOL 75 | 0.1 |
| Gluadin WQ | 0.2 |
| Gluadin WLM | 0.5 |
| Pantolactone | 0.5 |
| Subtilisin or papain | 0.5 |
| Carbopol | 0.1 |
| NEUTROL TE | 0.10 |
| ETHYL ALCOHOL DENATURED 96 VOL % | 30.0 |

pH = 7.0

### EXAMPLE 16

### Hair Tonic

| | |
|---|---|
| L-carnitine tartrate | 2.0 |
| Extract from Echinacea purpurea | 0.1 |
| Taurine | 0.01 |
| D-panthenol 75% | 0.20 |
| Allantoin | 0.10 |
| Perfume | 0.25 |
| Water, demineralized | ad 100 |
| Cremophor A25* | 0.2 |
| Ethanol 96% | 35.00 |

*Cremophor A25* = Ceteareth-25

### EXAMPLE 17

### Hair Rinse as in Example 1, as 2-Chamber System

1st chamber.

| | |
|---|---|
| Echinacea purpurea pressed juice (Flachsmann) | 0.6 |
| Eumulgin ® B2¹ | 0.3 |
| Cetyl/stearyl alcohol | 3.3 |
| Isopropyl myristate | 0.5 |
| Paraffin oil perliquidum 15 cSt. DAB 9 | 0.3 |
| Dehyquart ® A-CA² | 2.0 |
| Gluadin WQ | 0.2 |
| Gluadin WLM | 0.5 |
| Pantolactone | 0.5 |
| Phenonip ®³ | 0.8 |
| Water | ad 100 |
| pH = 4.0 | |

2nd chamber.

| | |
|---|---|
| L-carnitine | 2.0 |
| Taurine | 1.0 |
| Water | ad 100 |

### EXAMPLE 18

### Hair Treatment Analogous to Example 7, But in Two Application Steps

Pretreatment with L-carnitine tartrate and enzyme

| | |
|---|---|
| L-carnitine tartrate | 3.0 |
| Subtilisin or papain | 0.5 |
| or papaya extract | 1.0 |
| Water | ad 100 |

After-treatment.

| | |
|---|---|
| Sepigel ® 305 | 5.0 |
| Dow Corning ® Q2-5220 5 cSt.¹³ | 1.5 |
| Genamin ® DSAC¹⁴ | 0.3 |
| Phenonip ® | 0.8 |
| Gluadin WQ | 0.5 |
| Gluadin WLM | 0.8 |
| Pantolactone | 1.0 |
| Perfume oil | 0.25 |
| Taurine | 1 |
| Echinacea purpurea pressed juice (Flachsmann) | 0.5 |
| Water | ad 100 |

### EXAMPLE 19

### Hair Treatment (Leave On)

| | |
|---|---|
| Glyceryl monooleate | 0.50 |
| Methylparaben | 0.30 |
| Lactic acid 80% strength | 0.20 |
| Dehyquart A CA | 4.00 |
| Dehyquart L 80 | 5.00 |
| Pantolactone | 0.40 |
| Phenoxyethanol, pure | 0.50 |
| D-panthenol 75% | 0.20 |
| Nutrilan Keratin W¹⁷ | 0.50 |
| Dow Corning 1403 Fluid¹⁸ | 1.50 |
| Gluaden WLM | 0.50 |
| Perfume | 0.20 |
| Water | ad 100 |
| L-carnitine tartrate | 2.0 |
| Taurine | 1.0 |
| Echinacea purpurea pressed juice (Flachsmann) | 0.05 |

¹⁷Nutrilan Keratin W (22% strength active substance in water) = Keratin hydrolyzate (Cognis)
¹⁸Dow Corning 1403 fluid = Dimethicone, Dimethiconol (Dow Corning)

### EXAMPLE 20

### Determination of the ATP Synthesis Rate

ATP (adenosine triphosphate) is the universal storage form of chemical energy in cells. Cleaving off the distal phosphate group produces ADP and Pᵢ (inorganic phosphate). This reaction is highly exergonic, i.e., energy is released. ATP is produced during the cellular, oxidative degradation of fats, carbohydrates and proteins. It serves as energy supplier for biochemical syntheses, for transport processes (active transport) and for mechanical work. These processes are endergonic, i.e., they only proceed with the input of energy. In order to optimally maintain their metabolism, cells are thus reliant on an adequate supply of ATP. Dermal papillae cells also require ATP, for example, for the production of growth factors and thus for controlling the hair cycle. The proliferation and differentiation of ORS keratinocytes is likewise linked to ATP synthesis since the biosynthesis of specific proteins is an essential prerequisite for both processes. If the ATP synthesis rate of the hair-relevant cells can be increased by a product, then more energy is available to the cells in order to maintain metabolic processes and cellular structures and to renew structures, e.g., during repair processes or the new formation of hair.

ATP Detection Method.

The ATP determinations were carried out with the help of the ATP Lite TM-M assay (Packard). The test principle of this assay is based on the fact that the luciferase from Photinus pyralis catalyzes a reaction in which, in the presence of ATP, D-luciferin is converted to oxyluciferin. During this reaction, green light is emitted, which can be measured using a luminometer. The emitted bioluminescence light is proportional to the amount of ATP present.

To determine the ATP activity in dermal papilla cells, these are precultivated in a suitable manner while maintaining their specific properties (DE10162814) and transferred to a 48-well cell culture dish. Treatment with the substance mixture was carried out over 24 hours against an untreated control. The cells were then lysed with in each case 100 μl/cavity of a lyse buffer contained in the test kit for 5 min on a shaker. The cells were then incubated for a further 5 min with in each case 100 μl/cavity with the also supplied substrate solution on the shaker and then the reaction mixture was transferred to a black microtiter plate. After an incubation time of 10 min in the dark, the luminescence was measured.

The substance mixture of L-carnitine tartrate (0.01%), Echinacea purpurea [pressed juice from Echinacea purpurea (L.) Moench, Echinacea purp. hba succ. sicc, EFLA894, Flachsmann (article No. 008594) produced according to the method patented by Flachsmann (EP-B-0 730 830)] (0.1%) and taurine (0.01%) increased the ATP production of the dermal papilla cells by 65% compared with the untreated control (Table 1).

TABLE 1
Influence of the substance mixtures on the ATP production
of dermal papilla cells in % (standard deviation).

| | Mean |
|---|---|
| Untreated | 100 |
| Substance mixture | 165 (28) |

### EXAMPLE 21

### Detection of the Production of Hair-Relevant Growth Factors

Hepatocyte Growth Factor (HGF) and Keratinocyte Growth Factor (KGF) are important growth factors which are released from the dermal papillae in order to control the proliferation of the hair keratinocytes. A potentially growth-promoting and hair-strengthening substance can be assumed from an increase in the factors released into the medium. The release of HGF and KGF can be quantified with the help of commercially available ELISA kits. For this, organotypical cell cultures are incubated over 72 h with the substance mixture and the concentration of the growth factors in the medium is determined in the described manner.

The substance mixture of L-carnitine tartrate (0.01%), Echinacea purpurea [pressed juice from Echinacea purpurea (L.) Moench, Echinaceae purp. hba succ. sicc, EFLA894, Flachsmann (article No. 008594), produced according to the method patented by Flachsmann (EP-B-0 730 830)] (0.1%) and taurine (0.01%) increased the production of HGF compared with the untreated control by at most 412%, the production of KGF compared with the untreated control by at most 40%. Likewise detailed in Table 1 is the increase in the growth factors through application of the individual substances, the sum of which turns out lower than through the incubation of the mixture (Table 2).

TABLE 2
Influence of the substance mixture on the production of
growth factors [%](standard deviation).

| | HGF mean [%] | KGF mean [%] |
|---|---|---|
| Untreated | 100 | 100 |
| Substance mixture | 512 (6) | 140 (10) |
| Echinacea 0.1% | 445 (28) | 96 (9) |
| Taurine 0.01% | 120 (21) | 71 (7) |
| Carnitine tartrate 0.01% | 100 (42) | 108 (8) |

### EXAMPLE 22

### Increase in the Keratin Synthesis

The hair structure is essentially dependent on the composition of special hair-specific structure proteins, the hair keratins. By influencing the composition of these specific proteins it is possible to influence the hair structure on a biological level.

The expression of various hair keratins in the organotypical model can be investigated with the help of a quantitative Real-Time PCR process. To carry out the PCR, first the RNA is isolated from the organotypical models with the help of the RNeasy Mini Kit from Qiagen and transcribed into cDNA by means of reverse transcription. In the subsequent PCR reaction, which is carried out with the help of gene-specific primers for the particular hair keratins, and which serves to amplify the investigated gene segments, the formation of the PCR products is detected online via a fluorescence signal. The fluorescence signal here is proportional to the amount of PCR product formed. The greater the expression of a specific gene, the higher the amount of formed PCR product and thus the greater the fluorescence signal.

To quantify the gene expression, the untreated control is set as 1 and the expression of the genes to be determined is based on this (x-fold expression). Values which are greater than or equal to 1.8 times the expression of the untreated control are classified here as significant.

The substance mixture of L-carnitine tartrate (0.01%), Echinacea purpurea [pressed juice from Echinacea purpurea (L.) Moench, Echinacea purp. hba succ. sicc, EFLA894, Flachsmann (article No. 008594), produced according to the process patented by Flachsmann (EP-B-0 730 830)] (0.1%) and taurine (0.01%) increased the gene expression of the hair keratins hHa3-I and hHa4 in the organotypical model compared to the untreated control by a factor of 3.5 (Table 3).

TABLE 3
Influence of the substance mixture on keratin synthesis.

| | hHa3-I | hHa4 |
|---|---|---|
| Untreated | 1 | 1 |
| Substance mixture | 3.5 | 3.5 |

### EXAMPLE 23

### Detection of Various Hair-Relevant Markers by Means of DNA Array

In order to fully characterize the effect of the active ingredient mixture, organotypical cell cultures were treated for 6 h and 24 h with the active ingredient mixture of L-carnitine tartrate (0.01%), Echinacea purpurea [pressed juice from Echinacea purpurea (L.) Moench, Echinaceae purp. hba succ. sicc, EFLA894, Flachsmann (article No. 008594), produced according to the process patented by Flachsmann (EP-B-0 730 830)] (0.1%) and taurine (0.01%), the RNA was isolated and the expression of 850 different markers was investigated with the help of a DNA Chip Array. Untreated cell cultures were entrained as control. Both after 6 h and also after 24 h, a differential gene regulation was demonstrated for various hair-relevant parameters. Here, after 6 h, in particular, growth factors and proliferation, thus cell division, markers were highly regulated, after 24 h, those genes which point to an activation of the metabolism and also an induction of keratin synthesis and production of extracellular matrix (Table 4).

To quantify the gene expression, the untreated control is set as 1 and the expression of the genes to be determined is based on this (x-fold expression). Here, values which are greater than or equal to 1.7 times the expression of the untreated control are classed as statistically conspicuous, and values greater than or equal to 1.9 times the expression of the untreated control are classified as significant.

TABLE 4
Differential gene expression following treatment with the
substance mixture (untreated control = 1).

| | Expression 6 h | Expression 24 h |
|---|---|---|
| Growth/proliferation. | | |
| Ki 67 | 1.85 | −1.29 |
| IGF2 | 1.63 | −1.29 |
| cmyc | 2.02 | −1.28 |
| IER2 | 1.91 | −1.21 |
| EGF | −1.15 | 2.04 |
| Metabolism. | | |
| Glucose transporter type I | −1.07 | 2.00 |
| Hexokinase type II | −1.18 | 2.50 |
| Lactate dehydrogenase | −2.13 | 2.17 |
| Glutamate dehydrogenase II | −1.56 | 2.17 |
| Ornithin decarboxylase | 1.63 | 1.75 |
| Glutamin synthetase | 1.47 | 1.96 |
| Putative adenosylhomocysteinase | −1.20 | 2.27 |
| Extracellular matrix | | |
| Laminin alpha 2 | 1.42 | 1.85 |
| Laminin alpha 3 | 1.14 | 3.23 |
| Laminin gamma 2 | 1.08 | 1.92 |
| Collagen 11 | 1.07 | 2.44 |
| Collagen 17 | −1.6 | 3.45 |

The expression increase in the region of the growth factors and proliferation markers points to a hair-growth-promoting potential of the substance mixture, the increase in the expression of extracellular matrix genes points to a positive influence on the anchoring of the hair in the scalp since the hair follicle in the scalp is surrounded by collagen- and laminin-containing cell layers. Moreover, the stimulation of the growth factors also has a positive influence on the hair thickness since the hair thickness depends, inter alia, on the thickness of the ORS cell layer, which is responsible for the formation of the hair shaft. The increase in the metabolism activity also favors hair growth since building blocks such as, for example, amino acids for the formation of protein, have to be adequately provided for the underlying processes; the energy required for this is provided, for example, by the metabolism of glucose.

### EXAMPLE 24

### Increase in the Epthelium Thickness In Vitro

Since the growth factors HGF and KGF influence the proliferation of epthelium cells, the strengthening effect of the test substance used can also be demonstrated on the layer thickness of the model-supported ORS keratinocytes. For this, three sections are prepared from each of three organotypical models and measured at each of five points. To aid visibility, the histological sections are dyed beforehand with propidium iodide. With the help of a digital camera and image processing software it is possible to then measure the layer thickness of the individual models.

The substance mixture of L-carnitine tartrate (0.01%), Echinacea purpurea [pressed juice from Echinacea purpurea (L.) Moench, Echinaceae purp. hba succ. sicc, EFLA894, Flachsmann (article No. 008594), produced according to the process patented by Flachsmann (EP-B-0 730 830)] (0.1%) and taurine (0.01%) increased the epthelium thickness in the organotypical model compared with the untreated control by at most 80% (Table 3).

TABLE 5
Influence of the substance mixture on the epthelium thickness [%]
(standard deviation).

| | Epthelium thickness [%] |
|---|---|
| Untreated | 100 |
| Substance mixture | 180 (28) |

### EXAMPLE 25

### Thickening of the Hair Follicle In Vivo

To investigate the influence of the substance mixture on the thickness of the hair follicle in vivo, an in vivo study with 4 subjects was carried out. In a half-head test, a hair tonic comprising L-carnitine tartrate (2%), Echinacea purpurea [pressed juice from Echinacea purpurea (L.) Moench, Echinacea purp. hba succ. sicc, EFLA894, Flachsmann (article No. 008594), produced according to the process patented by Flachsmann (EP-B-0 730 830)] (0.05%) and taurine (1%) encapsulated in liposomes was compared with a placebo formulation without active ingredients (with liposomes). Before the start of the study, the starting value was determined for each subject on each side. To this end, in each case 6 hairs were plucked out and measured at three different points several times under standardized conditions. This measurement was repeated after one week and after two weeks in the same way.

After two weeks, in the case of one, a significant thickening of the follicle following treatment with the verum formulation compared with the half treated with placebo was demonstrated. Compared with the starting value, the hair follicle was thickened by around 20% in the region of the outer root sheath following application of the substance mixture (Table 6).

TABLE 6
Influence of the substance mixture on the thickening of the hair follicle
in the region of the outer root sheath [%] (standard deviation).

| | Starting value | 1 week | 2 weeks |
|---|---|---|---|
| Placebo | 100 (11) | 71 (8) | 80 (24) |
| Substance mixture (verum) | 100 (18) | 108 (21) | 120 (14) |

Example formulation of the hair tonic.

Verum formulation:
30% ethanol (cosmetic)
2% liposomes PC (Lipoid SL80, supplier Lipoid)
2% L-carnitine L-tartrate (supplier: Loncha)
0.05% Echinacea purpurea pressed juice (Flachsmann)
1% taurine
ad 100% water
   Placebo formulation:
30% ethanol (cosmetic)
2% liposomes PC (Lipoid SL80, supplier Lipoid)
68% water

### EXAMPLE 26

### Determination of Markers of the Thickening of the Hair Follicle In Vivo

To investigate the influence of an active ingredient mixture according to the invention on the thickness of the hair follicle, an in vivo study was carried out with fourteen subjects. In a half-head test, a leave-on treatment according to Example 19 comprising L-carnitine tartrate (2%), Echinacea purpurea (0.05%) [pressed juice from Echinacea purpurea (L.) Moench, Echinacea purp. hba succ. sicc, EFLA894, Flachsmann (article No. 008594), produced according to the process patented by Flachsmann (EP-B-0 730 830)] and taurine (1%) was compared with an untreated area on the upper part of the head.

The parameters investigated were the expression of the proliferation marker PCNA, and also the production of Hepatocyte Growth Factor (HGF) and Keratinocyte Growth Factor (KGF) on a protein level. HGF and KGF are growth factors with which the dermal papillae controls the growth and the proliferation of matrix keratinocytes. Matrix keratinocytes are localized in the bulb. From the thickness of the bulb, which is determined by the number of keratinocytes present, it is possible to draw conclusions about the follicle thickness and thus the thickness of the keratinized hair.

Before the start of the study, 15 hairs were pulled from all areas on the subjects and the expression of PCNA and also the production of HGF and KGF were determined. The products were applied 2× daily over a period of one week and further hair samples were removed for analysis every day in the corresponding areas.

Analysis of the PCNA expression revealed a significant advantage of the formulation comprising L-carnitine tartrate, Echinacea purpurea and taurine compared with the untreated area. This effect was detectable on day three and four.

TABLE 7
Relative change in expression through the active ingredient mixture
based on the untreated control (=1)

| | Day 1 | Day 2 | Day 3 | Day 4 |
|---|---|---|---|---|
| Untreated | 1 | 1 | 1 | 1 |
| Test formulation | −1.13 | 1.33 | 2.59 | 1.42 |

For better comparability of the release of protein, in each sample, the total protein content was additionally determined and the release of HGF/KGF was based on this (e.g., amount of KGF/μg of total protein). Compared with the starting value on day 0, quantification of the KGF production revealed an advantage of the formulation comprising L-carnitine tartrate, Echinacea purpurea and taurine compared with the untreated area at the end of the treatment time (day 4).

TABLE 8
Production of KGF [pg] based on the total protein, that stated
is the mean of all subjects with standard error of the mean (SEM).

| | Day 0 | Day 4 |
|---|---|---|
| Untreated | 2.5 (0.2) | 3.3 (0.6) |
| Test formulation | 3.3 (0.3) | 5.4 (1.7) |

Quantification of HGF led to no significant difference in the treated area.

1. A composition for the treatment of the hair or of the skin comprising L-carnitine or an L-carnitine derivative, the juice or an extract from the Echinacea plant, and taurine and/or an N-alkyl derivative of taurine.

2. The composition of claim 1 wherein the L-carnitine derivatives are selected from acetyl-L-carnitine, L-carnitine fumarate, L-carnitine citrate, lauroyl-L-carnitine and L-carnitine tartrate.

3. The composition of claim 1 wherein the composition is comprised of a combination of L-carnitine and L-carnitine derivative and an extract from the Echinacea plant, and taurine and/or an N-alkyl derivative of taurine.

4. The composition of claim 1 comprising from 0.001 to 10% by weight of L-carnitine or an L-carnitine derivative.

5. The composition of claim 1 comprising from 0.1 to 5% by weight, of L-carnitine or an L-carnitine derivative.

6. The composition of claim 1 comprising from 0.001 to 10% by weight of taurine and/or an N-alkyl derivative of taurine.

7. The composition of claim 1 comprising from 0.1 to 5% by weight of taurine and/or an N-alkyl derivative of taurine.

8. The composition of claim 1 wherein the amount of the juice or an extract from the Echinacea plant is from 0.001 to 10% by weight.

9. The composition of claim 8 wherein the amount of the juice or an extract from the Echinacea plant is from 0.01 to 2%.

10. The composition of claim 1 further comprising the juice or an extract from the Apiacea plant.

11. The composition of claim 10 wherein the Apiacea plant is Foeniculum vulgare.

12. The composition of claim 1 further comprising a hair-growth-stimulating active ingredient selected from the group consisting of finasteride and minoxidil.

13. A method of stimulating the growth of hair comprising contacting hair with a composition of claim 1.

14. A method for thickening eptheliumial cells and cell layers comprising contacting skin with a composition of claim 1.
